(19) United States
(12) Patent Application Publication
(10) Pub. No.: US 2023/0060466 A1
(43) Pub. Date: Mar. 2, 2023

(54) MEDICAL DEVICE AND METHOD FOR TREATMENT OF OSTEOARTHRITIS

(71) Applicant: Lance Liberti, Tampa, FL (US)

(72) Inventor: Lance Liberti, Tampa, FL (US)

(21) Appl. No.: 17/899,329

(22) Filed: Aug. 30, 2022

Related U.S. Application Data
(60) Provisional application No. 63/238,452, filed on Aug. 30, 2021.

(51) Int. Cl. A61K 31/728; A61F 5/01; A61K 9/00; A61K 35/16; A61K 35/19

(52) U.S. Cl. CPC A61K 31/728; A61F 5/0125; A61K 9/0019; A61K 35/16; A61K 35/19

(57) ABSTRACT

Combined therapy for the treatment of osteoarthritis of the knee. The therapy includes administration of medium to high molecular weight hyaluronic acid intraarticular injections followed by administration of platelet-rich plasma also administered by intraarticular injections. Before administration of the hyaluronic acid intraarticular injections, the knee can be stretched and exercised using a static progressive knee stretch device. The knee can be supported by a single hinge knee brace that can be worn daily, and during weight-bearing activity.

### CROSS-REFERENCE TO RELATED APPLICATIONS

This application claims priority from a U.S. Provisional Patent Appl. No. 63/238,452 filed on Aug. 30, 2021, which is incorporated herein by reference in its entirety.

### FIELD OF INVENTION

The present invention relates to a method for the treatment of osteoarthritis of knees, and more particularly, the present invention relates to a knee brace, a stretching device for a knee, and a method for the treatment of osteoarthritis of knees.

### BACKGROUND

Osteoarthritis (OA) is a form of arthritis that often affects the knee. According to the Kellgren Lawrence scale for the diagnosis of OA, the disease is broken down into five stages by which the joint experiences degeneration. Stage zero is documented by the absence of visible disease with no symptoms, though OA can be developing in secret. During the first stage of OA, there is no apparent narrowing of the knee joint to indicate cartilage loss, though cartilage loss has begun. Small lumps of bone called osteophytes may begin to grow on the knee, yet the knee will appear normal on an x-ray. When patients reach stage three of OA, the gaps between the bones are narrowed and the cartilage in the joint is significantly reduced or missing. As a result, the bones will grow denser to withstand the added pressure and load, causing thick areas of buildup to form lumps along the margin of the bones (subchondral sclerosis). This causes an inflammatory response within the synovial capsule, diluting the synovial fluid and further compromising the stability of the knee joint. Patients in this stage may find daily activities such as walking or bending exceedingly difficult.

Stage four OA is severe and is categorized by the absence of cartilage; creating a condition in which joint space becomes reduced and grinding of the bones against one another may occur. The large bone lumps that have formed make daily activities increasingly challenging, causing swelling and inflammation. Patients suffer a lower quality of life because of the pain and stiffness in the joints. At this stage, surgery is commonly recommended.

Osteoarthritis has been considered a modern health crisis, which affects more people today than any other type of arthritis. In fact, the CDC (2018) estimates that OA presently affects 32.5 million American adults, 88% of which are aged 45 and older. As a result of the crippling pain and stiffness associated with OA, obesity, heart disease, and mental health issues have high comorbidity rates. Comorbidities, lack of physical activity, and adverse effects of medication increase all-cause mortality in patients with OA by 55%.

Osteoarthritis is a progressive and degenerative disease with no known cure.

Symptoms of OA develop very slowly over years, and diagnosis does not typically occur until the patient is already experiencing moderate to severe disease progression. By that time, the disease is difficult to manage. Traditional treatments typically aim to mask the pain of the disease, and do not seek to target its underlying cause. Current treatment guidelines typically begin with non-pharmacologic protocols, such as patient education, weight loss, and physical therapy. Non-pharmacologic approaches frequently provide insufficient pain relief or restoration of function and mobility, necessitating pharmacologic modalities. Simple analgesics such as non-steroidal anti-inflammatory drugs (NSAIDs) provide symptomatic relief but are inappropriate in patients with gastrointestinal or cardiovascular risk factors.

Clinical research for osteoarthritis till now has largely ignored the interconnectivity of synovial fluid loss, ligament damage, muscle atrophy in the knee, joint deformity, knee alignment and their role in Osteoarthritis disease progression. The clinical research largely focused on the use of NSAIDs and/or steroid injections to control pain, though it has been widely accepted that long-term use of NSAIDs and/or steroid injections produce negative side effects and do not slow disease progression.

Thus, a long-term and unsolved need is there for treatment therapy for osteoarthritis, in particular for the osteoarthritis of knee.

The term osteoarthritis hereafter refers to osteoarthritis of knee.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a therapy for osteoarthritis that is safe and holistic.

It is another object of the present invention that the overall cost of treatment of osteoarthritis is significantly reduced.

It is still another object of the present invention that the therapy is nearly pain free.

It is a further object of the present invention that the need for knee replacement surgery can be avoided.

It is still a further object of the present invention that the therapy is minimally invasive.

In one aspect, disclosed is a method for treatment of osteoarthritis in patients in need thereof. The method includes implementing weekly intra-articular hyaluronic acid viscosupplementation knee injections using guided fluoroscopy for a total of five weeks, combined with a series of three intra-articular platelet-rich plasma (PRP) knee injections following the intra-articular hyaluronic acid injection on the first, third, and fifth visit, the daily use of a single hinge unloading knee brace, and the use of a static progressive knee stretch (SPS) device.

In one aspect, disclosed is a single hinge unloading knee brace.

In one aspect, the method includes determining candidacy for the disclosed therapy. The protocol dictates a rigorous patient consultation which includes obtaining the full medical history of a patient, documenting any prior failed treatments to address symptoms, completing of an arthrogram motion radiographic study, and synthesizing this knowledge in order to diagnose OA of the knee appropriately; identify the current stage of OA on the Kellgren and Lawrence Scale.

In one aspect, disclosed is a method that includes administering five weekly injections of a medium to high molecular weight hyaluronic acid (HA) viscosupplement directly into the knee joint capsule under guided fluoroscopy. Intra-articular knee injection with PRP drawn from the patient's blood can also be injected into the joint capsule by a qualified physician during the first, third, and fifth weeks of the treatment. HA and PRP combination treatment can significantly improve arthralgia, reduce humoral and cellular immune responses, and promote angiogenesis, which improves patients' histological parameters compared with PRP or HA treatment alone. Intra-articular injections of hyaluronic acid (HA) viscosupplementation and platelet-rich plasma (PRP) provide a better treatment option to address symptomatic pain, replenish the loss of synovial fluid in the joint space, encourage the body to produce higher levels of synovial fluid naturally, and delay the need for a total joint replacement surgery.

In one aspect, disclosed is a single hinge unloading knee brace and the use of the single hinge unloading knee brace in the treatment of osteoarthritis. The disclosed knee brace can be used on daily basis to support knee movement and limit any risky movement of the joint. The disclosed knee brace is advantageous by not immobilizing the knee and thus the user can perform prescribed physical activities and exercises easily. The disclosed knee brace can be comfortable to wear. In particular, the disclosed knee brace upon regular use can act by increasing the Tibia Femoral joint space, thereby reducing mechanical friction and destruction of meniscal and hyaline cartilage, without impeding joint range of motions.

In one aspect, disclosed is a knee stretch device and the use of the knee stretch device in the treatment of osteoarthritis. The use of the static progressive knee stretch (SPS) device allows the patients to increase joint extension and flexion safely and easily, leading to overall better range of motions and decreasing the stiffness and unsteadiness associated with OA of the knee. The SPS device also improves blood flow to the area, accelerating the healing mechanisms associated with HA and PRP therapy. Lastly, the SPS device causes strengthening of the muscles and tendons around the joint to protect it. The static progressive knee stretch device allows patients to simulate the range of motion and stretch exercises that physical therapists often perform during traditional OA knee rehabilitation therapy.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a general knee joint.

FIGS. 2a and 2b depict the difference in a knee joint six months following the treatment, FIG. 2a is before and FIG. 2b is after, according to an exemplary embodiment of the present invention.

FIG. 3 is a front view of a Knee Brace worn over a knee joint, according to an exemplary embodiment of the present invention.

FIG. 4 is a perspective view of the knee brace, according to an exemplary embodiment of the present invention.

FIG. 5 shows a static progressive knee stretch device worn over the leg and operated by a user, according to an exemplary embodiment of the present invention.

FIG. 6 demonstrates the widening of the Tibia Femoral joint space after using the disclosed knee brace, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

Disclosed are a knee brace, a static progressive knee stretch device, and a method for the treatment of osteoarthritis. Disclosed are the devices and methods for knee joint rehabilitation using combinatorial therapy. The disclosed devices and methods provide for safe, effective, and minimally invasive therapy. The disclosed method combines medical therapy that includes medium to high molecular weight intra-articular hyaluronic acid viscosupplementation and platelet-rich plasma (PRP) injections, and a physical therapy that includes the use of disclosed knee brace and the disclosed static progressive knee stretch device to decrease pain in both during and after rehabilitation therapy. The disclosed method provides for an increase in proprioception during and after rehabilitation therapy and fortifies the anti-inflammatory response both in OA damaged compartments of the knee and the unaffected compartments. Reduction of pain and inflammatory response allows for an increase in joint stability and a range of motions, and further can significantly delay or avoid the need for knee replacement surgery.

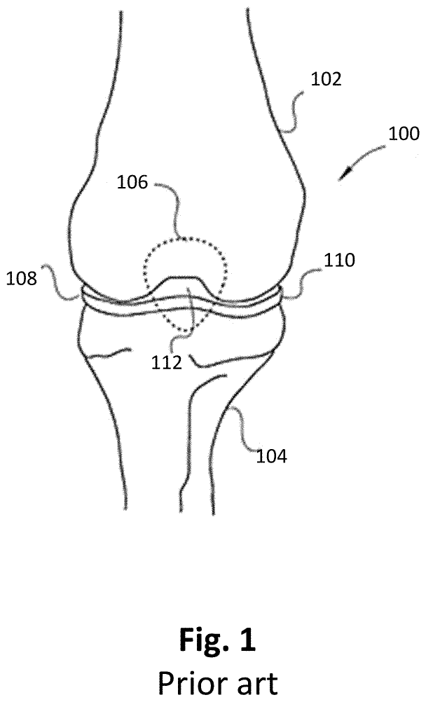

FIG. 1 illustrates the anatomy of a typical knee joint 100 that has three knee compartments. A femur 102, a tibia 104, and a patella 106 that form a knee joint 100. The femur 102 has two distinct protrusions at its lower (distal) end, known as the lateral condyle and the medial condyle. These provide two weight-bearing surfaces between the tibia 104 and the femur 102. The space formed by the weight-bearing surfaces on the outer portions of the knee 100 is a first compartment 108 which corresponds to the lateral tibial femoral joint space. The space between the weight-bearing surfaces on the inner portions of the knee 100 is a second compartment 110, which corresponds to the medial femorotibial compartment. A third compartment 112 is in the center of the knee, formed between the patella 106 and the space between the lateral and medial condyle of the femur 102, which corresponds with the patellofemoral compartment.

In a normal knee joint 100, the first compartment 108, the second compartment 110, and the third compartment 112 include synovial fluid, endogenous hyaluronic acid, articular cartilage, and the medial and lateral meniscus all of which help to protect the weight-bearing surfaces of the femur 102 and the tibia 104 during movement. Hyaluronic acid is an unbranched polysaccharide made up of alternating glucuronic acid and N-acetylglucosamine units, called glycosaminoglycan (GAG). Hyaluronic acid is a viscoelastic material that is important for building a component of aggregated proteoglycans which result in the resilient characteristics of articular cartilage.

When the knees are affected by osteoarthritis, the articular cartilage of the first compartment 108, the second compartment 110, the third compartment 112, or the combination begins to degrade along with gradual loss of its extracellular matrix (ECM), which is composed of aggrecan and type II collagen. Loss of proteoglycan aggrecan decreases the stiffness of cartilage which leads to a decrease in the separation between the weight-bearing surfaces causing pain. Intra-articular injection of hyaluronic acid weekly for five weeks reduces the friction that occurs in degenerating knee joints and increases the spacing between the femur and the tibia. Intra-articular hyaluronic acid injections protect against enzymes that further break down articular cartilage and act as an anti-inflammatory agent within the joint. Intra-articular platelet-rich plasma (PRP) injections completed on weeks one, three, and five, following the injection of hyaluronic acid release cytokines, growth factors, and other bio-active molecules into the joint space, improving the repair of articular cartilage injury and strongly reducing the level of inflammatory factor present in the synovial fluid.

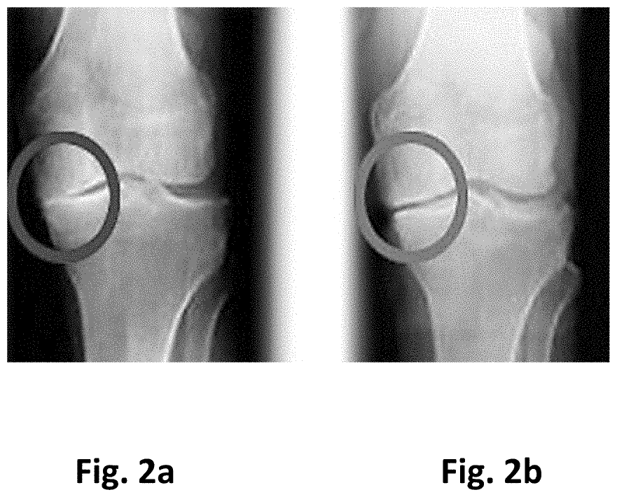

FIG. 2 depicts the visible difference in the health of a knee joint six months following the five-week therapy, highlighted in FIG. 1, largely by the increase in the tibial femoral joint space. Prior to the start of treatment, a full patient medical history can be obtained, and an arthrogram can be performed using a C-arm fluoroscopic X-ray machine in order to accurately diagnose the stage of OA and determine candidacy for the disclosed treatment. Fluoroscopic technology allows for viewing of the bones as well as other structures, such as cartilage. Contrast medium such as iopamidol or ioxaglate meglumine can be injected into the intra-articular space so that the knee joint can be properly observed on the fluoroscope. The contrast medium has no effect on the health of the joint and carries no side effects unless the patient is allergic to it. The arthrogram provides a three-dimension view of the joint by capturing anterior, posterior, and lateral views while the knee rests at a 90-degree angle. The joint is a suitable candidate for the disclosed therapy if OA of the knee is diagnosed and the knee capsule is intact, without synovial tears. A therapy including a series of five 2.5 ml intra-articular hyaluronic knee injections and three intra-articular PRP injections can then be administered with the assistance of a contrast medium and guided fluoroscopy to ensure accuracy. The compartment of the knee within the joint capsule that is deemed by the physician and patient to be the source of the most damage/pain should be targeted first. The hyaluronic acid viscosupplement with a medium to high molecular weight greater than or equal to 500 kilo Daltons (kD) is then injected intra-articularly. A medium to high molecular weight hyaluronic acid is comprised of long chain molecules and is understood to provide higher levels of joint cushioning and pain reduction. The medium to high molecular weight hyaluronic acid viscosupplement gets metabolized by the body, however, it has a long-term effect compared to the lower molecular weight counterparts. The slower metabolic process allows for maximum opportunity for joint healing and re-lubrication.

Intra-articular platelet-rich plasma injections in the volume of 2 ml or greater can be administered following the viscosupplementation injection on the first, third, and fifth weeks of treatment. PRP is completely autologous, meaning that it is derived from the patient's own body, and therefore, safe with no contraindications. HA and PRP combination treatment significantly improves arthralgia, reduces humoral and cellular immune responses, and promotes angiogenesis, which improves patients' histological parameters compared with PRP or HA treatment alone. Intra-articular injections of hyaluronic acid (HA) viscosupplementation and platelet-rich plasma (PRP) provide a better treatment option to address symptomatic pain, replenish the loss of synovial fluid in the joint space, encourage the body to produce higher levels of synovial fluid naturally, and delay the need for a partial or total joint replacement surgery (PTKA/TKA).

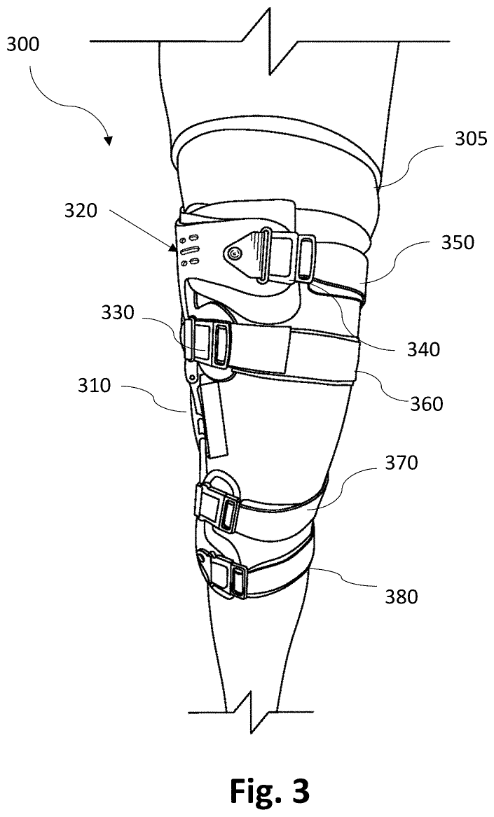

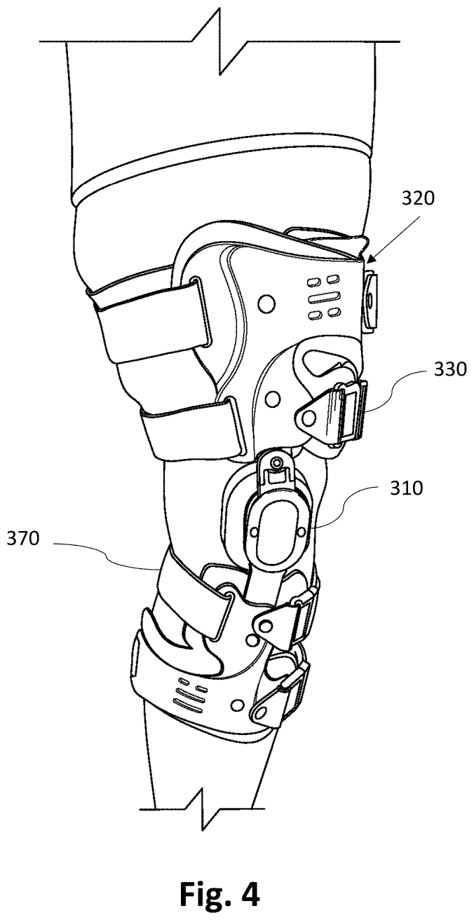

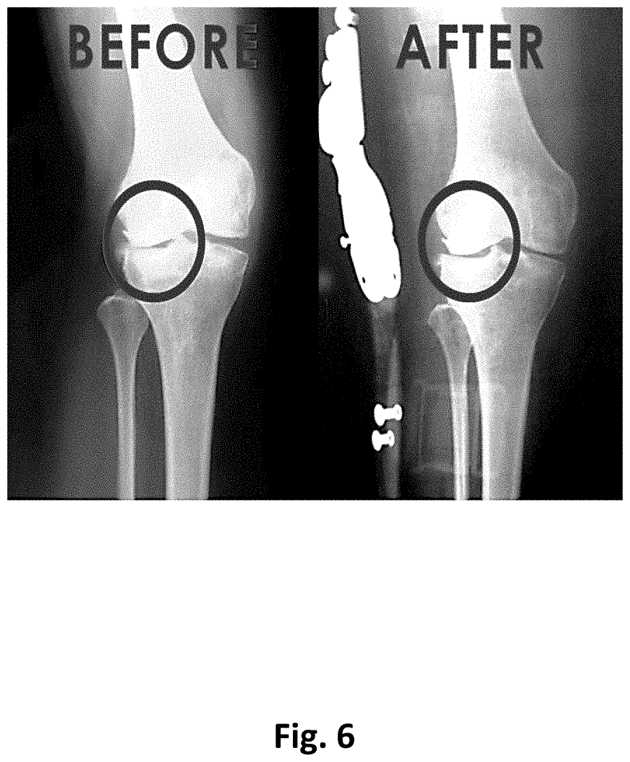

FIGS. 3 and 4 depict an exemplary embodiment of a Single Hinge Unloading Knee Brace 300. FIG. 6 demonstrates the widening of the Tibia Femoral joint space after bracing with the Knee Brace even before the hyaluronic acid and PRP treatment has begun. The unloading knee brace is fitted for the patient at the start of treatment and should be worn daily during any weight-bearing activities throughout the course of treatment.

The disclosed knee brace can include a single hinge that provides for the desired degree of immobilization and movements for physical therapy. The disclosed single hinge knee brace can have an advantage over the traditional dual hinge knee braces, a typical dual hinge knee brace provides relief in pain by immobilizing the knee, however, the further restriction of movement is counterproductive to the commonly prescribed physical therapy regimen given to OA patients and can actually expedite joint rigidity; causing further damage to the ligaments, tendons, and other soft tissue of the knee in the long term. The disclosed single hinge knee brace overcomes the drawbacks of the traditional double hinge knee brace.

The disclosed therapy is risk-free, drug-free treatment option to retard joint degeneration, relieve painful OA symptoms, complement PT treatment plans, and reduce the incidence of knee replacement surgery. The disclosed single hinge unloading Knee brace can be comfortable and cost-effective. The single hinge unloading Knee brace can increase the tibia femoral joint space, thereby reducing mechanical friction and destruction of meniscal and hyaline cartilage, without impeding joint range of motion. An orthosis suspension sleeve can also be incorporated into the design to provide better fitting for patients with comorbid obesity. The sleeve gently compresses the adipose tissue of the thigh, two inches above the patella of the knee so that the superior portion of the brace may contact the femur bone, thereby providing appropriate anchoring for unloading of the Tibia Femoral joint space.

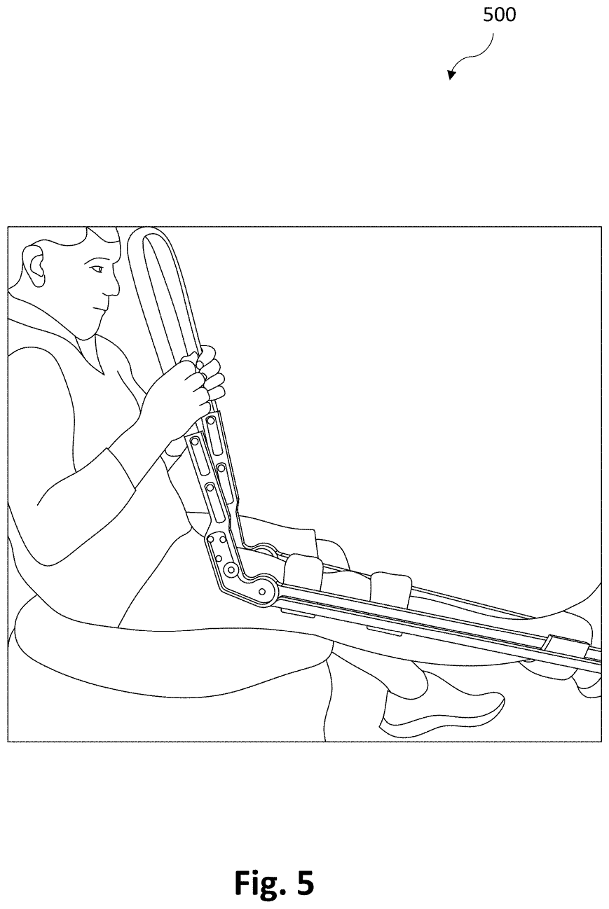

FIG. 5 demonstrates the use of the static progressive knee stretch device (SPS) 500. The SPS device simulates the flexion and extension exercises commonly prescribed by physical therapists and can be performed daily in the comfort of the patient's home. Moreover, the SPS device is designed to be used in a seated position, reducing the risk of injury or fall when compared to traditional physical therapy exercises. The SPS device complements the other combinatorial therapies of the HA, PRP, and knee brace by increasing blood flow to the joint, accelerating the healing mechanisms associated with HA and PRP injections. The exercises performed with the SPS device also strengthen the muscles and tendons around the joint, protecting the joint, and improving the overall joint function and steadiness. Flexion and Extension exercises outlined in the easy-to-follow instructions of the SPS device can be performed daily and prior to intra-articular injections on injection days. Weight-bearing movement and SPS exercises can be avoided immediately after intra-articular injections for a minimum of two hours following the injection(s). The SPS device also serves to assist clinicians by providing another tool for measuring joint functionality and improvements in activities of daily living (ADL's).

A case study of treated patients revealed a 92.7% success rate, indicating a 50% or more reduction in pain, and 50% or more improvement in mobility. The treatment protocol is minimally invasive, poses virtually no risks, and addresses the root cause of Osteoarthritis and progressive loss of synovial fluid.

The disclosed single hinge unloading knee brace 300 can be worn over an orthosis suspension sleeve 305 to provide better fitting for patients with comorbid obesity. The sleeve can gently compress the adipose tissue of the thigh two inches above the knee so that the superior portion of the brace may contact the femur bone, thereby providing appropriate anchoring for unloading of the Tibia Femoral joint space. One in three patients diagnosed with obesity may develop symptomatic knee osteoarthritis in their lifetime. The disclosed knee brace was evaluated in an outpatient clinical setting with osteoarthritis patients receiving an intra-articular injection treatment protocol. Patient outcomes were compared with patients who were not using a brace or were using a dual hinge brace. Based on the outcomes, it was found that the single hinge knee brace design was superior. Disclosed knee brace specifically addresses and treats knee osteoarthritis. Moreover, one size can fit most patients and does not need to be tailored for each individual. This saves both the cost and time, that the patient does not have to wait for the custom braces.

The disclosed knee brace includes an iron sub-frame 320 and a hinge 310 that can be sturdy enough to not degrade under extraordinary pressure resulting in an impressive lifespan of 3-5 years or longer when being used during all weight-bearing activities. Rivets can be used at pivot points instead of screws that can come loose or break. Durable Velcro® straps 350, 360, 370, and 380 and plastic clips 330 and 340 are implemented for proper fitting of the brace. The knee brace can be cushioned with marine grade memory foam to avoid skin irritation and resist wear from exposure to sweat and other biological factors. The knee brace features a slim design that fits comfortably under pants and so has better patient compliance. Flexion and extension range of motion limiting stops are included in the accompanying hardware bag in the event, the patient is also having ligament or tendon injury. In such cases, the flexion and extension range of motion limiting stops are set to the desired degree to inhibit the range of motion so as to prevent exacerbation of or impede the healing of said soft tissue injury. Extension stops range from 0-30 degrees and flexion stops range from 50-100 degrees. Upon approval, the range of motion stops may be removed to allow for a full range of motion once again. Two Allen keys can also be included with the knee brace that may allow one to adjust the varus-valgus set rivet, and one for removal of the hinge cap in order to apply a flexion and extension range of motion limiting stop, if required.

In one exemplary embodiment, the disclosed knee brace can deliver pain relief and improved quality of life for patients suffering from osteoarthritis of the knee. Tibia Femoral joint space can be increased by using the disclosed knee brace to unload pressure on the cartilage of the knee and knee capsule and retard further joint degeneration. The disclosed knee brace can also provide protection against soft tissue damage through flexion and extension stops, when necessary.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for treatment for osteoarthritis of a knee, the method comprising:
administering, medium to high molecular weight hyaluronic acid, through intra-articular injection; and
upon administering the medium to high molecular weight hyaluronic acid, administering platelet-rich plasma, through intra-articular injection.

2. The method according to claim 1, wherein the platelet-rich plasma is administered in alternate weeks.

3. The method according to claim 1, wherein the medium to high molecular weight hyaluronic acid is administered weekly for five consecutive weeks and the platelet-rich plasma is administered on 1^{st}, 3^{rd}, and 5^{th} week.

4. The method according to claim 1, wherein the medium to high molecular weight hyaluronic acid and the platelet-rich plasma are administered under guided fluoroscopy.

5. The method according to claim 1, wherein the method further comprises:
supporting the knee using a single hinge unloading knee brace, wherein the single hinge unloading knee brace comprises only one hinge.

6. The method according to claim 5, wherein the single hinge unloading knee brace is worn over an orthosis suspension sleeve.

7. The method according to claim 5, wherein the method further comprises:
stretch exercising the knee before administering the medium to high molecular weight hyaluronic acid.

8. The method according to claim 7, wherein the method further comprises:
providing a static progressive knee stretch device for stretch exercising.

9. The method according to claim 1, wherein the medium to high molecular weight hyaluronic acid and platelet-rich plasma are delivered in a compartment of the knee where damage/pain is determined to be the highest.

10. The method according to claim 1, wherein the medium to high molecular weight hyaluronic acid has a molecular weight of more than 500 kDa.

11. The method according to claim 6, wherein the single hinge unloading knee brace is worn during any weight-bearing activity.
